(19) United States
(12) Patent Application Publication
(10) Pub. No.: US 2015/0359889 A1
(43) Pub. Date: Dec. 17, 2015

(54) Cardiac arrest muscle relaxant

(71) Applicant: Michael Lewis Moravitz, Fairfax, VA (US)

(72) Inventor: Michael Lewis Moravitz, Fairfax, VA (US)

(21) Appl. No.: 14/756,204

(22) Filed: Aug. 17, 2015

(51) Int. Cl. A61K 45/00

(52) U.S. Cl. CPC A61K 45/00

(57) ABSTRACT

Muscle Relaxants for Cardiac Arrest Patients

Muscle Relaxants might be developed for cardiac arrest patients that could be used instead of or with current treatments.

After a seizure, perhaps a doctor or EMT could apply a muscle relaxant and then CPR and defibrillation if necessary. Relaxing the heart and then restarting may improve survivability instead of just a sudden shock to the system. Also ice packs to heart attack victims are supposed to cool the heart down but may in fact cause severe shock. Cool water may be better, especially to the skin of the patient instead of to the heart itself. Heart tissue is very sensitive. My ideas, of course, would need to be researched and backed up by experiences of medical professionals themselves before being implemented.

1. Previous Patents for Muscle Relaxant Medicines.
